(19) United States
(12) Patent Application Publication
(10) Pub. No.: US 2016/0089118 A1
(43) Pub. Date: Mar. 31, 2016

(54) Urine Specimen Collection Device

(71) Applicant: Michelle L. Petersilia, Plano, TX (US)

(72) Inventor: Michelle L. Petersilia, Plano, TX (US)

(21) Appl. No.: 14/496,423

(22) Filed: Sep. 25, 2014

(51) Int. Cl. A61B 10/00

(52) U.S. Cl. CPC A61B 10/007

(57) ABSTRACT

A urine specimen collection device that aids in positioning a standard urine specimen cup in a urine flow path to avoid inadvertent contact between the user's hands and the urine being collected. A standard specimen collection cup is insertable through an aperture disposed in a cup holder portion of the device. The aperture is preferably expandable from a first size to a larger second size when a specimen cup is inserted and is preferably configured to support the cup in a position where around 50% or more of the height of the cup is disposed on an underside of the cup holder body. The cup holder end of the device may bend at an angle between about 120° and 170° relative to a handle end of the device when a specimen cup is inserted through the aperture. Optional handle extensions may be used to further elongate the device.

### BACKGROUND OF THE INVENTION

1. Field of the Invention

This invention relates to a handheld urine specimen collection device that aids in positioning a standard urine specimen cup in a urine flow path substantially under the urethra of the person providing the sample so that the user's hands may be maintained a distance away from the urethra to avoid inadvertent wetting or contact between the user's hands and the urine being collected.

2. Description of Related Art

Urine samples are a commonly used medical diagnostic tool. Most typically, obtaining a sample of urine requires the sample provider or another person (such as a home health aide, nurse, or family member), to use their hands to position a specimen cup under the sample provider's urethra to collect the urine sample within the specimen cup. It may be difficult to properly position the specimen cup under the sample provider's urethra because of difficulty reaching the proper position, particularly when the person holding the specimen cup is not the sample provider. Additionally, the hand holding the specimen cup may be inadvertently wetted by the urine during the collection process. This can be a nuisance for the person holding the cup and considered unsanitary or unhealthy. Perhaps more importantly, if the urine contacts the person's hands and it is then deposited in the specimen cup, the urine sample may be contaminated by such contact which may adversely impact the diagnostic results.

There are several known devices to aid in urine specimen collection. Many of these involve funnel-like configurations that are positioned to catch a flow of urine and direct it to a collection container located a short distance from the sample provider, such as that disclosed in U.S. Pat. Nos. 4,559,649, 4,457,314, 5,622,183, 5,735,835, 6,702,793, 6,151721, 7,993,312. Similar funnel-like devices configured to attach to a specimen container are disclosed in U.S. Patent Nos. 3,161,891 and 5,125,118 and U.S. Patent Application Publication No. 20140135651. A more complicated, multi-chamber device is disclosed in U.S. Pat. No. 5,409,473. A device that attaches to a toilet is disclosed in U.S. Pat. No. 5,146,637. Many of these devices still require a user to manually position the funnel portion of the device under the sample provider and require contact between the device and the sample provider's body, which may result in contamination. At least two of these devices, U.S. Pat. Nos. 6,151,721 and 5,735,835, disclose the use of adhesive or tape to secure the device to the sample provider's body, which may be painful to remove. All of the known devices require the urine sample to flow through at least some portion of the device. To ensure the urine sample is not contaminated by external contaminants, these devices should be sterilized prior to use. Additionally, the devices are not ergonomically designed to allow easy positioning of a standard specimen collection cup under the urethra of the sample provider, particularly when the person collecting the sample (and positioning the cup) is someone other than the provider. There is a need for a simple urine specimen collection holder that may be used with standard specimen cups, does not require the urine to flow through any part of the holder device, and allows for ease of collection.

### SUMMARY OF THE INVENTION

The urine specimen collection holder device disclosed herein may be used with standard specimen collection cups, to support the cup and aid in positioning the cup under the urethra of the person providing the urine sample (referred to herein as the sample provider) and in the flow of urine. According to one preferred embodiment, the device comprises a handle and a cup holder. The cup holder comprises an aperture configured to receive a specimen cup and hold the specimen cup so that it is partially disposed through the cup holder and supported by the cup holder. The overall length of the handle and cup holder are preferably between about 9 and 14 inches long. This enables the user (the user may be the sample provider or another person, such as a nurse, home healthcare aid, or family member) to position the specimen cup while maintaining his or her hands a distance away from the flow of urine to avoid contact between the user's hands and the urine for sanitary purposes and to avoid contaminating the urine sample. One or more optional handle extensions may also be used to further lengthen the device, which may be particularly useful if the user is someone other than the sample provider.

According to another preferred embodiment, a plurality of slits and cooperating support members are disposed around the aperture in the cup holder. The slits and support members cooperate to form an opening for receiving the specimen cup that is larger than the aperture. When a specimen cup is inserted into the aperture, the supporting members are forced downward to create the larger opening.

According to yet another preferred embodiment, the plurality of slits comprises slits of different lengths, which cooperate to form supporting members of different sizes.

A crease is disposed through a cup holder in yet another preferred embodiment. This crease allows the cup holder to be folded back over the handle to reduce the size of the device for packaging, shipping, and storage purposes. The crease also aids in allowing a portion of the cup holder to bend relative to the handle when a specimen cup is inserted. This bend creates an angle, preferably of between about 120° and about 170° relative to a longitudinal axis through the handle, which aids in the ergonomics of the device and in comfortably positioning the device in the urine flow path.

The preferred embodiments of the invention have several advantages over the known prior art. These include that the use of a handle makes positioning the device easier and allow the user's hands to be located a sufficient distance from the specimen cup and urine flow path to avoid contact between the urine and the user's hands, which makes use of the device more sanitary and avoids contaminating the specimen. A longer device and the inclusion of optional handle extensions also make the device easier to use when the user is someone other than the sample provider. There is no need for any adhesives to hold the device or specimen cup in position and it is not necessary for either the device or the specimen cup to contact the sample provider's body during collection. Although it is preferred to sterilize the device prior to shipping the device for use, sterilization is not required since the urine specimen does not flow through or contact any part of the device before it is collected in the specimen cup so there is no risk of contaminating the specimen through contact with the device. The device is also less complicated and easier to use than prior art devices. It is preferably made of biodegradable materials so that it may be disposed of easily and in an environmentally friendly manner and may be toilet flushable.

### BRIEF DESCRIPTION OF THE DRAWINGS

The preferred embodiments of the invention are further described and explained in relation to the following drawings wherein:

FIG. 1 is a perspective view of a urine specimen collection device according to a preferred embodiment of the invention;

FIG. 2 is a side elevation view of the urine specimen collection device of FIG. 1, shown with a specimen cup in place in the cup holder;

FIG. 3 is a perspective view of an optional handle extension for the urine specimen collection device according to one embodiment of the invention;

FIG. 4 is an end elevation view of the handle extension of FIG. 3;

FIG. 5 is a top plan view of a cup holder according to another embodiment of the invention;

FIG. 6 is a top plan view of a cup holder according to another embodiment of the invention; and

FIG. 7 is a perspective view, partially broken, of a urine specimen collection device according to a preferred embodiment showing a handle extension attached to a handle.

### DESCRIPTION OF THE PREFERRED EMBODIMENTS

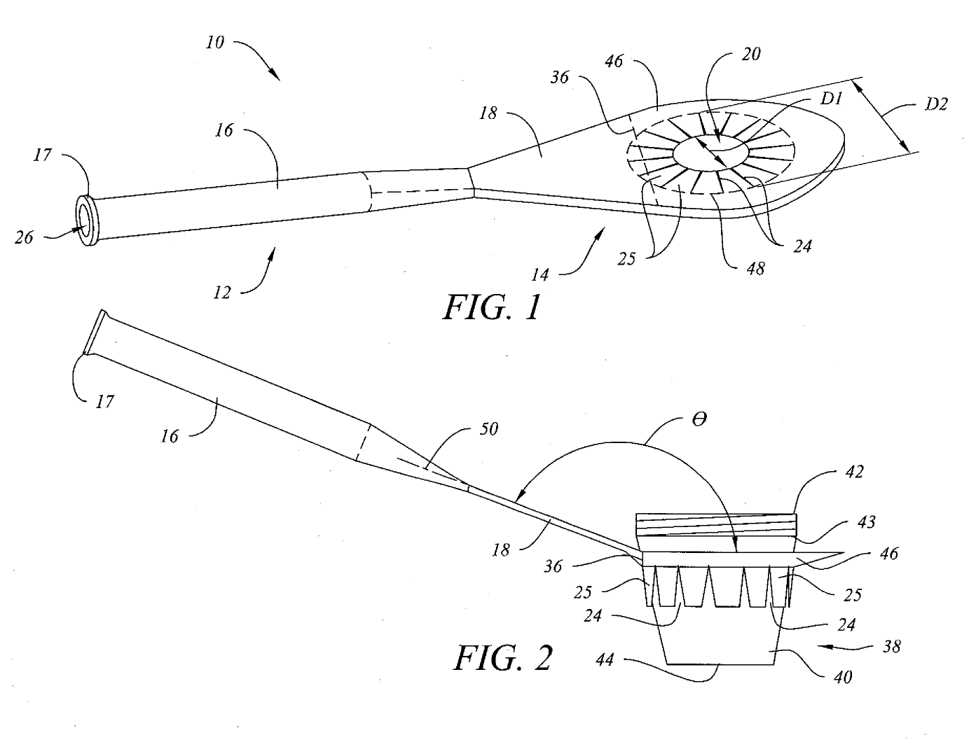

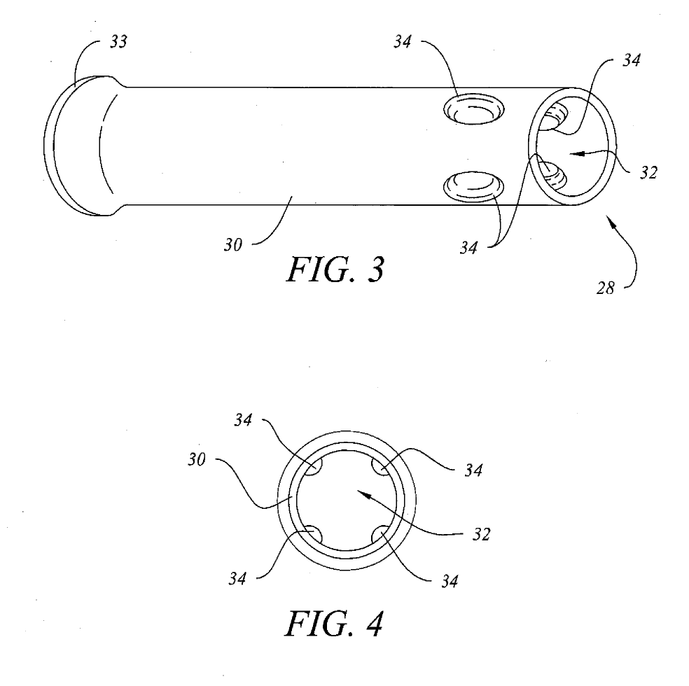

One preferred embodiment of a urine specimen collection device 10 according to the invention is depicted in FIGS. 1-4. Urine specimen collection device 10 preferably comprises a handle 12, a cup holder 14, and optionally one or more handle extensions 28 (shown in FIGS. 3-4). Handle 12 preferably comprises a handle body 16 that attaches at a front end to a rear end of cup holder 14. Cup holder 14 preferably comprises a cup holder body 18, an aperture 20 disposed through cup holder body 18, a plurality of slits 24 disposed around and extending radially outward from an outer perimeter of aperture 20 and plurality of flexible support members 25, each disposed between a pair of slits 24. The aperture 20, slits 24, and support members 25 cooperate to allow a specimen cup, such as cup 38 shown in FIG. 2, to be inserted into aperture 20 and be supported by cup holder 14.

A specimen cup 38, used to collect a urine sample is shown in FIG. 2. Cup 38, which is a standard, commercially available specimen cup and not one specially designed for use with the invention, typically has a substantially cylindrical body 40 with a sealed lower end 44 and an upper end 42 configured to receive a removable lid that keeps the interior of body 40 clean and sterile during shipping and storage prior to use and seals the urine sample inside between deposit of the sample and analysis of the sample. Most preferably upper end 42 comprises threads that mate with threads on the lid. The cylindrical body 40 tapers slightly from the threaded end 42 toward the bottom end 44. Although there may be some variation in size, usually the diameter of the cup 38 at upper end 42 is around 1.75-2.5 inches and the diameter at the lower end 44 is around 1.5-2 inches. Preferably, aperture 20 is circular or substantially circular with a diameter D1 around 0.6 to 1.2 inches for a specimen cup 38 having a lower end 44 with a diameter of around 1.5-2 inches. Most preferably, the diameter D1 of aperture 20 is around 40-60% of the diameter of the lower end of the specimen cup with which urine specimen collection device 10 will be used.

A plurality of slits 24 cut through cup holder body 18 are disposed around aperture 20. Most preferably, between 10 and 40 slits 24 are provided and are substantially equally spaced around aperture 20. Slits 24 are preferably between 0.5 and 1.0 inches long and more preferably between 0.7 and 0.8 inches long. Slits 24 are preferably pre-cut through body 18 for ease of use in positioning a specimen cup 38 within cup holder 14. Perforations may also be used, which allow slits 24 to be fully opened upon insertion of a specimen cup within cup holder 14. A plurality of flexible supporting members 25 are formed between each pair of slits 24. When a specimen cup 38 is inserted into aperture 20, the larger size diameter of lower end 44 forces supporting members 25 to flex downward, creating a larger opening 48 (the outer perimeter of which corresponds to the radially distant ends formed by slits 24 and is shown in dashed lines in FIG. 1) through which most of specimen cup body 44 may be inserted. This larger opening 48 is preferably circular or substantially circular and has a diameter D2 around 1.6 to 2.6 inches for a specimen cup 38 having an upper end 42 with a diameter of around 1.75 to 2.5 inches. Most preferably, the diameter D2 of opening 48 is around 60% to 110% of the diameter of the upper end of the specimen cup with which urine specimen collection device 10 will be used. A substantially same size or larger size for D2 compared to the diameter of the upper end of the specimen cup may be used when cup 38 includes a lip 43 or similar protrusion that effectively gives it a larger diameter near the upper end of the cup and may also be used because supporting members 25 cooperate to form an opening (disposed around the lower ends of support members 25 as shown in FIG. 2) to receive cup body 40 that is slightly smaller than D2 so that specimen cup 38 is supported by cup holder 14.

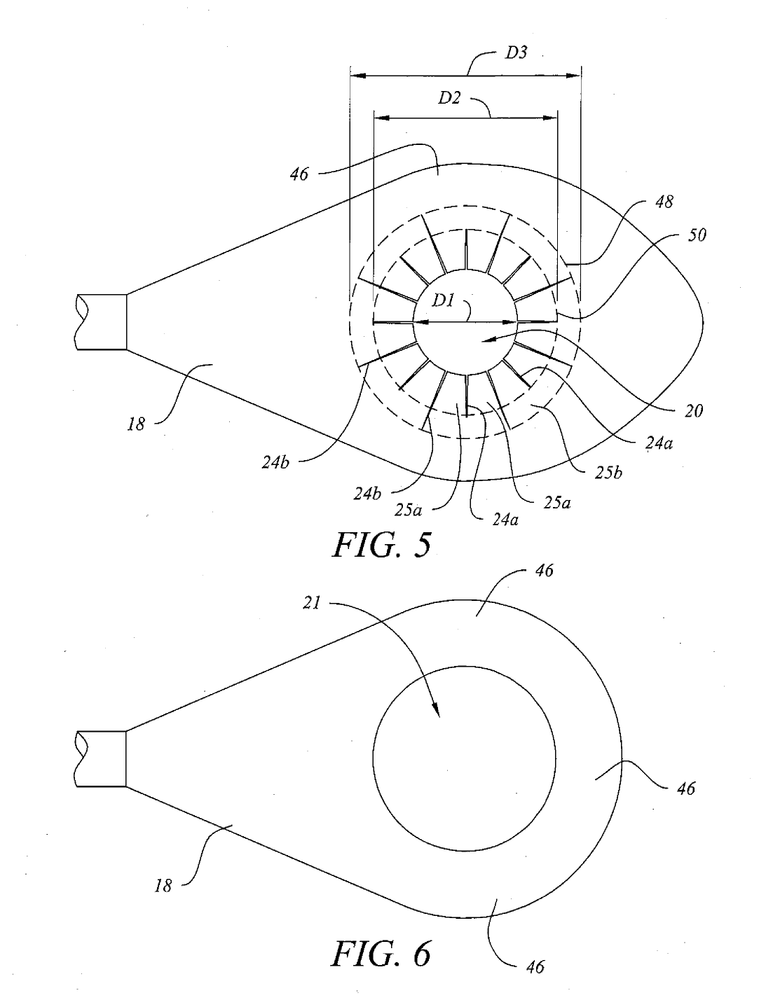

A lip 46 is disposed around the right, left, and front sides of opening 48. When a specimen cup 38 is inserted into urine specimen collection device 10, a lip 43 formed just below or formed by a lower thread on upper end 42 of specimen cup 38 may or may not contact lip 46 on cup holder 14, depending on the amount of force applied by the user and the dimensions of slits 24 and opening 48 relative to the specific specimen cup used. Lip 46 is preferably sized to accommodate the upper end 42 of specimen cup 38, to further support specimen cup 38 in cup holder 14 if lip 43 of specimen cup 38 contacts lip 46. Lip 46 may also provide strength and stability for cup holder 14, to prevent the weight of the specimen cup 38 with a urine sample from causing cup holder body 18 to tear, particularly at or near a radially outward end of a slit 24 or if cup holder body 18 is wetted during collection of the specimen. Most preferably lip 46 extends radially outward from opening 48 by around 0.25-0.5 inches or more. Cup holder body 18 may be substantially circular or ovoid in shape. If an ovoid shape is used, as depicted in FIGS. 1 and 5, then lip 46 may be larger at the front end than on the sides of opening 48. When a specimen cup 38 is inserted into cup holder 14, most preferably, around 50% or more of the height of specimen cup 38 will be disposed under a plane even with lip 46.

As shown in FIG. 5, slits 24 may optionally comprise different lengths. Shorter slits 24a may extend radially outward from an outer perimeter of aperture 20, having a length of around 0.3 to 0.7 inches, preferably around 0.5 inches. Longer slits 24b may extend radially outward from an outer perimeter of aperture 20, having a length of around 0.5 to 1.0 inches, preferably around 0.75 inches. Most preferably, the slits 24a, 24b alternate every other slit or with two shorter slits 24a disposed between each pair of longer slits 25a. When different lengths are used, support members 25 are segmented into shorter segments 25a and longer segments 25b. It is preferred to use different lengths for slits 24a, 24b to provide greater flexibility for support members 25a, 25b while maintaining overall strength in the supporting structure. The radially distant ends of shorter slits 24a form a circular or substantially circular shape 50 (shown in dashed lines in FIG. 5) having a diameter D3 of around 1.2 to 2.2 inches, for the specimen cup size described herein. Most preferably, D3 is around 70% to 95% of the diameter of the upper end of the specimen cup with which device 10 will be used.

Slits 24 (or 24a and 24b) and supporting members 25 (or 25a and 25b) cooperate to effectively enlarge aperture 20 from a first diameter D1 when no specimen cup is inserted into cup holder to a second, larger diameter, which is preferably larger than the diameter of the bottom end 44 of specimen cup 38 but smaller than the diameter of lip 43 (measured from an outer edge of annular lip 43 across to the 180° opposite outer edge) or smaller than the diameter of upper end 42 (if no lip 43 is present). When slits 24 and supporting members 25 are used, this second, larger diameter is the diameter formed by the lower ends of support members 25 when flexed downward by insertion of a specimen cup, as shown in FIG. 2. Most preferably, this second, larger diameter is sized between D2 and D3, or around 70-98% of the diameter of the upper end of the specimen cup with which urine specimen collection device 10 will be used, depending on how far into the cup holder 14 a specimen cup is inserted, the force of insertion applied by the user, and the size of the specimen cup.

Although the use of slits 24 and supporting members 25 are preferred, cup holder body 18 may also comprise a larger aperture 21 without utilizing a slit and supporting member configuration (as shown in FIG. 6). Such a larger aperture 21 would be sized to fit a specimen cup through the aperture, preferably so that at least a lower half of the height of specimen cup body 40 is disposed below a plane even with cup holder body 18. Such aperture 21 may be around the diameter of opening 48 or sized between the diameters D2 and D3, as previously described. A lip 46 would be disposed around the right, left, and front sides of aperture 21. When a specimen cup 38 is inserted into urine specimen collection device 10, lip 43 of specimen cup 38 may or may not contact lip 46 on cup holder 14, depending on the amount of force applied by the user and the dimensions of aperture 21 relative to the specific specimen cup used. Most preferably, the largest diameter of aperture 21 is slightly smaller than the diameter of lip 43 on specimen cup 38 (or slightly smaller than the diameter of the upper end of the cup if the cup does not include a lip), so that specimen cup 38 may be supported by lip 46 if device 10 is configured to allow lip 43 to contact lip 46. FIG. 6 also shows a more rounded embodiment for the front end of cup holder body 18, where lip 46 is substantially the same size on the sides of aperture 21 and to the front of aperture 21.

Handle body 16 preferably comprises a substantially cylindrical hollow tube. A distal end of body 16 (relative to cup holder 14) comprises an opening 26 configured to mate with an optional handle extension, such as extension 28. An optional lip 17 may project radially outward from body 16 at the distal end of body 16. A rear end of cup holder 14 preferably extends into an interior portion of handle body 16, with the proximal end of handle body 16 preferably being crimped and secured to a rear end of cup holder 14 by adhesive or other conventional attachment mechanism. Other configurations for handle body 16, such as a substantially flat rectangular shape may also be used. Additionally, handle body 16 and cup holder body 18 may be unitarily formed. Most preferably, handle body 16 is sufficiently long, so that when coupled with the preferred length of cup holder 14, a specimen cup may be easily placed under the urethra of the sample provider so that the user's hands do not need to contact the sample provider's body or come in contact with the urine while the specimen is being deposited in the specimen cup. Most typically, the urine specimen collection device 10 is used to obtain a sample from a sample provider positioned in a seated position (such as seated on a toilet with the collection device 10 being inserted into an area of the toilet bowl between the sample provider's legs), or in a squat or slight squat position, or in an standing position with one leg elevated (such as one leg elevated onto a toilet seat or other surface and one leg on the floor).

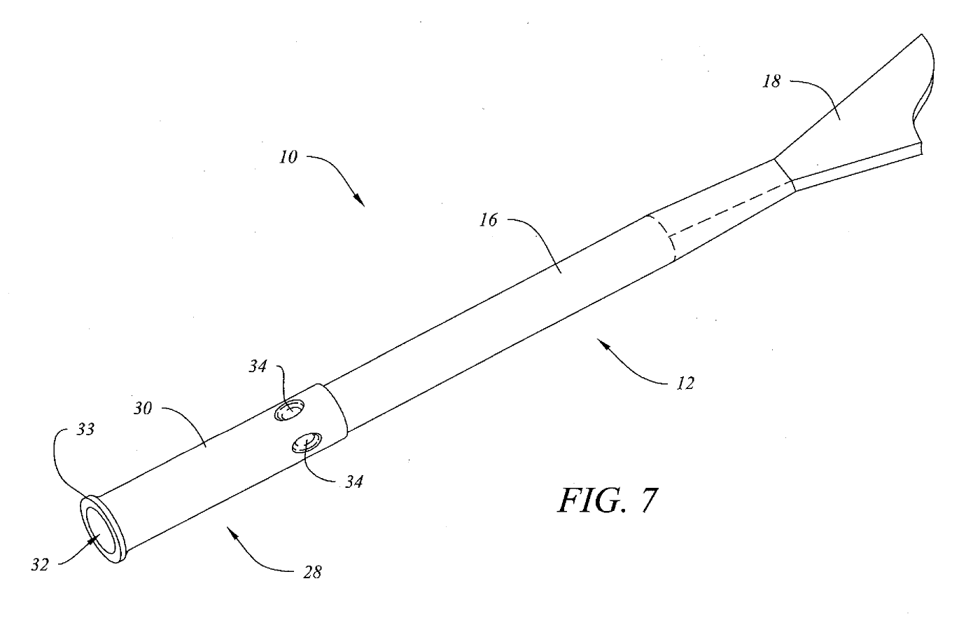

As shown in FIGS. 3-4 and 7, one or more optional handle extensions 28 may also be used with device 10. Each handle extension preferably comprises an extension body 30 and a plurality of projections 34 disposed near an end of body 30 configured to connect to handle body 16. Body 30 is preferably a substantially cylindrical tube comprising an opening 32. An optional lip or similar protrusion 33 may project radially outward from body 30 at a distal end of body 30 (relative to cup holder 14). Projections 34 project inwardly into an interior of body 30 as shown in FIGS. 3-4. In this configuration, handle body 16 is inserted into opening 32 and projections 34 cooperate to create a smaller diameter area in opening 32 to hold handle body 16 in position by friction (as shown in FIG. 7). If optional lip 17 is provided on handle body 16, it may be forced rearwardly of projections 34 to further aid in holding handle body 16 in position relative to handle extension 28. Alternatively, projections 34 could project outwardly from body 30 with body 30 configured to be inserted into opening 26 on handle body 16. As an additional alternative, projections similar to 34 may be disposed on body 16, either projecting inwardly or outwardly depending on whether body 30 is inserted into opening 26 or body 16 in inserted into opening 32. Additional optional extension handles may be similarly configured and inserted into each other in series to obtain a desired overall length of device 10 to properly position it under the sample provider's urethra. The use of one or more handle extensions 28 may be particularly useful to obtain extra length for device 10 if the user is someone other than the sample provider. Other configurations for optional extension body 30 and handle body 16 may also be used, such as a substantially rectangular body with an opening 32 or 26 at only one end, a tapered end configured for a friction or compression fit rather than the use of projections, a clip disposed at the end of either body 30 or body 16 configured to attach to the other body, or a threaded connection between body 30 and body 16. As an additional alternative, handle extensions may be configured to telescope from the handle.

To properly position the specimen cup under the sample provider's urethra, it is preferred that handle body 16 be around 5-8 inches long and that cup holder body 18 be around 4-6 inches long when the user is the sample provider. To properly position the specimen cup under the sample provider's urethra, it is preferred that one or more handle extensions 28 totaling around 2.5 to 5 inches in length, or more, be added to the handle body 16 when the user is someone other than the sample provider. Other lengths for handle body 16, cup holder body 18, and optional extensions 28 may also be used, regardless of whether the user is the sample provider, depending on various factors, such as the body size of the sample provider and the position from which the sample is obtained.

Cup holder body 18 preferably comprises a crease shown by dashed line 36 in FIG. 1. Crease 36 allows cup holder body 18 to be folded back over handle 12 to reduce the size of device 10 for purposes of packaging, shipping, and storage. Crease 36 also provides a break-line for cup holder body 18 or a portion of cup holder body 18 disposed forwardly of crease 36 to bend when a specimen cup 38 in inserted into device 10. This creates an angle ⊖ between an upper surface of cup holder body 18 disposed forwardly of crease 36 and the remainder of cup holder body 18 and handle body 16, relative to a longitudinal axis 50 through handle body 16. Angle ⊖ is preferably obtuse, more preferably between about 120° to 170° and most preferably around 140°. This angle aids in positioning the specimen cup under the sample provider's urethra so that the position of handle body 16 is more ergonomic for the user. This angle is particularly useful when the sample is being obtained from a seated position on a toilet, so that the device 10 can be positioned between the sample provider's legs while still allowing the handle body 16 to clear the toilet seat. Crease 36 is preferably located to bisect opening 48 rear of a centerline through opening 48 (disposed side to side of opening 48), but may also be positioned rearward of opening 48 or at a point where cup holder body 18 is attached to handle body 16.

In the preferred embodiments shown in FIGS. 1-3, the diameter of cup upper end 42 is around 2.3125 inches, the diameter of cup lower end 44 is around 1.8 inches, lip 43 protrudes from body 40 by 2-3 mm, the diameter of aperture 20 is around 0.875 inches, the length of each slit 24 is around 0.75 inches, the diameter of larger opening 48 is around 2.375 inches, lip 46 is around 0.375 inches to the sides of aperture 20 and around 0.75 inches at its longest point to the front of aperture 20, cup holder body 18 is around 4.75 inches long and around 3.0625 inches wide at its widest point, handle body 16 is around 5.625 inches long, the outside diameter of opening 26 is around 0.4375 inches, handle extension 28 is around 2.625 inches long, outside diameter of opening 32 is around 0.625 inches, and protrusions 34 project inwardly by about 2 mm.

Cup holder 14, handle 12, and handle extensions are most preferably made of cardboard or paperboard materials so that they are biodegradable. Preferably, these materials are coated with a wax coating or type of coating that increases the strength of device 10 and improves on resistance to wetting during sample collection, as wetting may decrease the strength of device 10 when made of cardboard or paperboard. The types of materials typically used for cardboard tampon applicators may be used. Other materials, such as plastics, may also be used for device 10. Preferably cup holder 14, handle 12, and handle extensions 28 are made of the same material. Alternatively, cup holder 14, handle 12, and/or extensions 28 may be made of different materials. For example, handle 12 and extensions 28 may be made of cardboard and cup holder 14 may be made of plastic material that would be resistant to wetting, as cup holder body 18 is more likely to be wetted during sample collection than handle 12 or extensions 28. In that configuration, handle 12 may be removable from cup holder 14, so that the biodegradable and non-biodegradable materials may be disposed of in a manner appropriate to the different materials.

References to front (or forward) and rear (or back) herein generally refer to the direction when urine specimen collection device 10 is being used, with cup holder 14 disposed at the front end of device 10 toward the sample provider. Reference to up (or upward or the like) and down (or downward and the like) herein generally refer to the direction in which device 10 is used when a specimen cup 38 is in position within device 10, where upward is the openable/lid end of specimen cup 38 and downward is the sealed bottom end of specimen cup 38. Those of ordinary skill in the art will also appreciate upon reading this specification and the description of preferred embodiments herein that modifications and alterations to the apparatus and methods may be made within the scope of the invention and it is intended that the scope of the invention disclosed herein be limited only by the broadest interpretation of the appended claims to which the inventor is legally entitled.

We claim:

1. A urine specimen collection device comprising:
a handle; and
a cup holder disposed forwardly of the handle, the cup holder comprising an aperture configured to receive and support a specimen cup while a urine specimen is being collected.

2. The urine specimen collection device of claim 1 wherein the cup holder further comprises a plurality of slits disposed around and extending radially outward from the aperture and a plurality of supporting members, each supporting member disposed between a pair of slits.

3. The urine specimen collection device of claim 1 wherein a substantial portion of a forward end of the cup holder is configured to bend when a specimen cup is inserted into the aperture to form an angle of between about 120° and about 170° relative to a longitudinal axis through the handle.

4. The urine specimen collection device of claim 2 wherein a substantial portion of a forward end of the cup holder is configured to bend when a specimen cup is inserted into the aperture to form an angle of between about 120° and about 170° relative to a longitudinal axis through the handle.

5. The urine specimen collection device of claim 1 wherein the handle and cup holder have a combined length between about 9 to about 14 inches.

6. The urine specimen collection device of claim 1 further comprising a handle extension, configured to add length to the handle.

7. The urine specimen collection device of claim 6 wherein the handle extension is configured to telescope from the handle.

8. The urine specimen collection device of claim 6 wherein the handle extension is separate from the handle and is releasably attachable to the handle.

9. The urine specimen collection device of claim 6 wherein the handle extension, handle and cup holder have a combined length between about 11.5 and about 19 inches.

10. The urine specimen collection device of claim 1 wherein urine does not flow through the device to reach the specimen cup.

11. The urine specimen collection device of claim 2 wherein the plurality of slit comprises one or more slits having a first length and one or more slits having a second length, wherein the first length is longer than the second length.

12. The urine specimen collection device of claim 2 wherein slits and support members cooperate to enlarge the aperture from a first diameter when no specimen cup is inserted in the cup holder to a second larger diameter when a specimen cup is inserted in the cup holder.

13. The urine specimen collection device of claim 12 wherein the first diameter is around 40% to 60% of a diameter of a bottom end of the specimen cup with which the device is to be used and the second diameter is around 70% to 98% of a diameter of an upper end of the specimen cup with which urine specimen collection device 10 will be used.

14. The urine specimen collection device of claim 6 wherein the handle comprises a substantially tubular body, the handle extension comprises a substantially tubular body and wherein the outer diameter of one of the tubular bodies is slightly smaller than the other tubular body so that the smaller diameter tubular body may be inserted into the larger diameter tubular body to connect the handle and handle extension by friction.

15. The urine specimen collection device of claim 14 wherein the larger diameter tubular body comprises a plurality of inwardly projecting protrusions that aid in holding the smaller diameter tubular body in position relative to the larger diameter tubular body.
